(19) United States
(12) Patent Application Publication
(10) Pub. No.: US 2011/0230707 A1
(43) Pub. Date: Sep. 22, 2011

(54) PELVIC IMPLANT SYSTEM AND METHOD

(76) Inventors: Jessica L. Roll, Minnetonka, MN (US); James A. Gohman, Minnetonka, MN (US); Michael J. Rossing, Minnetonka, MN (US); John F. Otte, Minnetonka, MN (US); Gregory L. Koeller, Minnetonka, MN (US)

(21) Appl. No.: 13/063,319

(22) PCT Filed: Sep. 15, 2009
(86) PCT No.: PCT/US2009/056935

§ 371 Date: May 2, 2011

Related U.S. Application Data
(60) Provisional application No. 61/097,106, filed on Sep. 15, 2008.

(51) Int. Cl. A61F 2/00

(52) U.S. Cl. 600/37

(57) ABSTRACT

A pelvic Implant system is provided. The system can include one or more implant devices (11) to treat incontinence and other pelvic floor disorders or dysfunctions. The system can include one or more implant devices having an extension portion (12) (e.g., mesh), one or more tip portions (14,16) (e.g., self-fixating tips), and one or more intermediate anchors (18a, 18b) disposed along the extension portion, intermediate the one or more tip portions. The intermediate anchors are shaped and sized for engagement with the internal pelvic tissue, with the extension portion in turn providing support for the corresponding pelvic tissue.

### RELATED APPLICATION

This application claims priority to and the benefit of U.S. Provisional Application No. 61/097,106, filed Sep. 15, 2008, which is incorporated herein by reference in its entirety.

### FIELD OF THE INVENTION

The invention relates to systems and methods for treating pelvic conditions and, more particularly, to an implant device to secure to and/or support pelvic tissue.

### BACKGROUND OF THE INVENTION

Pelvic health for men and women is a medical area of increasing importance, at least in part due to an aging population. Examples of common pelvic ailments include incontinence (fecal and urinary) and pelvic tissue prolapse (e.g., female vaginal prolapse). Urinary incontinence can further be classified as including different types, such as stress urinary incontinence (SUI), urge urinary incontinence, mixed urinary incontinence, among others. Other pelvic floor disorders include cystocele, rectocele, enterocele, and prolapse such as anal, uterine and vaginal vault prolapse. A cystocele is a hernia of the bladder, usually into the vagina and introitus. Pelvic disorders such as these can result from weakness or damage to normal pelvic support systems.

Urinary incontinence can be characterized by the loss or diminution in the ability to maintain the urethral sphincter closed as the bladder fills with urine. Male or female SUI occurs when the patient is physically stressed.

The female's natural support system for the urethra is a hammock-like supportive layer composed of endopelvic fascia, the anterior vaginal wall, and the arcus tendineus. Weakening and elongation of the pubourethral ligaments and the arcus tendineus fascia pelvis, and weakening of the endopelvic fascia and pubourethral prolapse of the anterior vaginal wall, may have a role in the loss of pelvic support for the urethra and a low non-anatomic position that leads to urinary incontinence.

In general, urinary continence is considered to be a function of urethral support and coaptation. For coaptation to successfully prevent or cure incontinence, the urethra must be supported and stabilized in its normal anatomic position. A number of surgical procedures and implantable medical devices have been developed over the years to provide urethral support and restore coaptation.

One alternative surgical procedure is a pubovaginal sling procedure. A pubovaginal sling procedure is a surgical method involving the placement of a sling to stabilize or support the bladder neck or urethra. There are a variety of different sling procedures. Although complications associated with sling procedures are infrequent, they do occur. Complications include urethral obstruction, prolonged urinary retention, bladder perforations, damage to surrounding tissue, and sling erosion.

Elongated fixating slings have also been introduced for implantation in the body, to treat pelvic conditions such as prolapse and incontinence conditions. Various systems and methods sold by American Medical Systems, Inc. under the product names BioArc® and SPARC® provide a single use sling implantation tools sold in a kit with an elongated urethral sling.

Another known implant system includes the use of a sling device having a self-fixating tip at a distal end of an extension portion, and is sold under the product name MiniArc® by American Medical Systems, Inc. The self-fixating tip can be placed at and secured within internal tissue of the pelvic region to support the implant end extension and pelvic tissue that is supported by the implant. As an example, a self-fixating tip can be placed at tissue of the obturator foramen (this phrase referring to tissue that lies within or spans the obturator foramen, for example the obturator internus muscle, the obturator membrane, or the obturator externus muscle). Embodiments of these self-fixating tips can be designed to provide desired function and performance in positioning and tissue attachment. For example, a self-fixating tip can be designed to provide desirably low input force, desirably high pullout force, and reduced trauma caused by passage of the self-fixating tip or an associated insertion tool.

### SUMMARY OF THE INVENTION

The present disclosure describes pelvic implant systems, devices and methods for treating pelvic conditions such as incontinence (e.g., fecal incontinence, stress urinary incontinence, urge incontinence, mixed incontinence, etc.), vaginal prolapse (e.g., enterocele, cystocele, rectocele, vault prolapse, etc.), and other like conditions or dysfunctions. Embodiments of various implant devices including one or more tips, or self-fixating tips, at a generally distal end of one or more extension portions, and one or more intermediate anchors generally attached, integrated or otherwise provided with the extension portion. The extension portion can be constructed of a mesh or woven polymer or like compatible material.

The one or more anchors along portions of the extension portion can be referred to as intermediate anchors provided in one embodiment proximate the ends of the mesh extension portions, or otherwise intermediate the end device tips, to provide or increase fixation until tissue in-growth into the implant occurs. In another embodiment, the one or more intermediate anchors can be disposed in one or more positions along a length of the mesh extension portions.

In one embodiment, the tips can be self-fixating tips securable within internal tissue of the pelvic region to further assist in supporting the implant device. The one or more intermediate anchors and tips can be configured of various sizes and shapes. The tips of the implant can be designed to engage a distal end of an insertion tool to allow the insertion tool to place the tip and intermediate anchors at a desired tissue location via pushing.

Embodiments of the intermediate anchors and tips can be designed to provide desired fixation while simultaneously reducing trauma caused by passage of the implant and the corresponding insertion tool through and into the pelvic region. These functional properties can result from selecting desired overall dimensions (length or width) for the anchors and tips, angles of the anchor structure, linear or curvature designs, and other size, shape and extension configurations.

In one embodiment, the invention provides a method of treating urinary incontinence in male and female patients (e.g., SUI) in a minimally invasive manner including injecting a local anesthetic; creating only one medial (e.g., transvaginal) incision under the mid-urethra; inserting a urinary incontinence sling implant through the one transvaginal incision, anchoring the urinary incontinence sling, and closing the incision.

Another aspect of the invention includes a combination (e.g., kit, system, etc.) of an implant device, as described herein, including one or more fixating tips and one or more intermediate anchors. The kit also includes one or more insertion tools or systems useful for inserting, positioning and deploying the implant device.

In another aspect, the invention relates to a method of treating a pelvic condition. The method includes providing an implant device according to the current description; providing an insertion tool that includes a handle and a needle extending from the handle, the needle including a proximal end attached to the handle and a distal end, the distal end including a needle distal end that removably or selectively engages the device tip; engaging the needle distal end with the tip; inserting the needle distal end and tip through an incision in a patient; and inserting the tip and corresponding one or more intermediate anchors into tissue in the pelvic region such that an extension portion of the implant device supports the targeted pelvic tissue.

### BRIEF DESCRIPTION OF THE DRAWINGS

Other features and advantages of the present invention will be seen as the following description of particular embodiments progresses in conjunction with the drawings.

FIG. 1 is a view of a pelvic implant system, having an insertion tool and implant device, in accordance with embodiments of the present invention.

FIG. 2 is a view of an implant device having end tips, an extension portion and intermediate anchors, in accordance with embodiments of the present invention.

FIG. 3 is schematic cross-section view of an implant device having end tips, an extension portion and intermediate anchors, in accordance with embodiments of the present invention.

FIG. 4 is a partial cross-section view of an implant device, showing the extension portion and an intermediate anchor, in accordance with embodiments of the present invention.

FIG. 5 is a partial view of an implant device having an end tip portion coupled or in communication with an extension portion, in accordance with embodiments of the present invention.

### DETAILED DESCRIPTION OF THE INVENTION

The following description is meant to be illustrative and not limiting. Other embodiments of this invention will be apparent to those of ordinary skill in the art in view of this description, claims and corresponding figures.

The present invention is directed to surgical instruments, assemblies, and implantable articles for treating pelvic floor disorders such as fecal or urinary incontinence, including stress urinary incontinence (SUI), prolapse, etc. According to various embodiments, a surgical sling or implant device can be used to treat a pelvic condition, including the specific examples of implanting a support implant to treat a condition such as vaginal vault prolapse or incontinence (male or female).

The sling implant or system may include portions or sections that are synthetic (e.g., polymer) or constructed of biological material (e.g., porcine, cadaveric, etc.). Extension portions may be constructed of a synthetic mesh such as a polypropylene, or other like materials. Examples of implant devices and tools that may be useful according to and with the present description include those sold commercially by American Medical Systems, Inc., of Minnetonka Minn., under the trade names Apogee® and Perigee® for use in treating pelvic prolapse (including vaginal vault prolapse, cystocele, enterocele, etc.), and Sparc®, Bioarc®, Monarc® and MiniArc®, for treating urinary incontinence. U.S. Pat. Nos. 6,911,003, 6,612,977, 6,652,450, 2009/0192347, 2008/0119863, 2008/0045782, and 2004/0039453, and International PCT Publication No. 2008/057261, disclose various implant devices, structures, procedures, systems and methods or techniques capable of use with the present invention and are, therefore, incorporated fully herein by reference.

Embodiments of the present invention, as shown in FIGS. 1-5, include a sling implant system 10 that can be installed to help maintain continence by supporting the urethra during times of increased abdominal pressure. The present invention also includes methods of implanting the sling. The sling system 10 can be implanted through a single incision in the vaginal wall for females (transvaginally), or perineal floor for males, and attached to (e.g., anchored) the obturator internus muscle on either side of the urethra. Only requiring one incision in the vaginal wall (for females) or perineum (for males) eliminates additional incisions such as external incisions used in some methods of implanting urethral slings. The sling system 10 and its methods of implantation can be, therefore, a reduced or “minimally” invasive treatment option for patients suffering from urinary incontinence.

In various embodiments, the sling system 10 may be anchored at other locations besides the obturator internus muscle, such as, for example, the obturator membrane, the obturator externus muscle, etc.

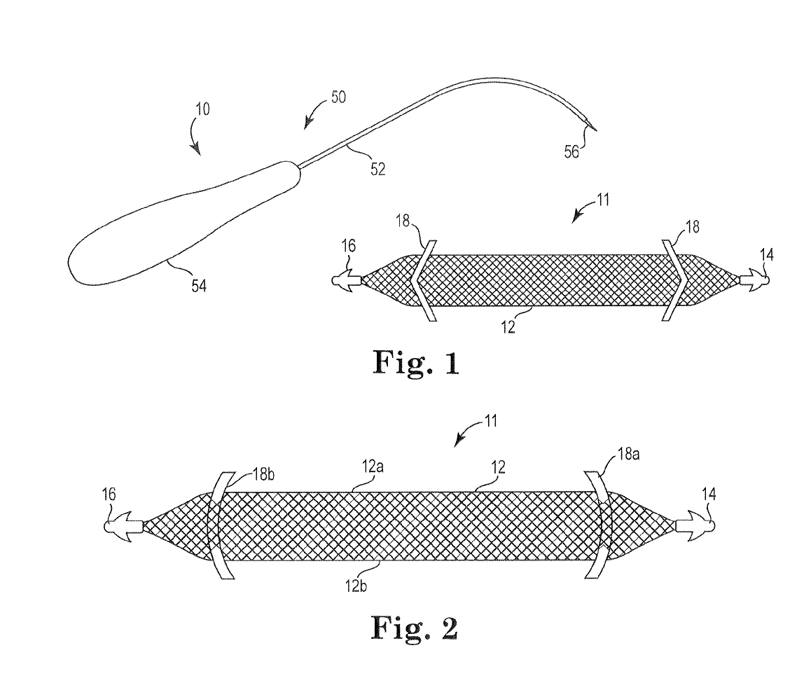

Referring to FIGS. 2-5, the implant system 10 can include an implant device 11 having an elongate extension portion 12, end or tip portions 14, 16, and one or more intermediate anchors 18. The extension portion 12 can include a first generally longitudinal side 12a and a second generally longitudinal side 12b. The longitudinal sides 12a, 12b can extend a distance between the end or tip portions 14, 16.

The extension portion 12 can be constructed of a polymer (e.g., polypropylene) mesh material, or other materials known for use with incontinence slings or pelvic tissue support devices. The extension portion 12 may be woven, knitted, sprayed, solid, or punched from a blank. In one aspect of the invention, extension portion 12 may include one or more woven, knitted, or inter-linked filaments or fibers that form multiple fiber junctions. In addition, the size of the resultant openings or pores of a mesh embodiment of the extension portion 12 may be sufficient to allow tissue in-growth and fixation with surrounding tissue. Additionally, the extension portion 12 may be surface coated or impregnated with epithelialization-promoting agents, drugs or other materials to enhance tissue impregnation.

Further, the extension portion 12 can include an intermediate band portion 22. The band portion 22 can be a plasma-treated print area of the sling extension portion 12 or a separately coupled or integrated band or indicia. The portion 22 can facilitate tracking of the device during the surgical implant procedure.

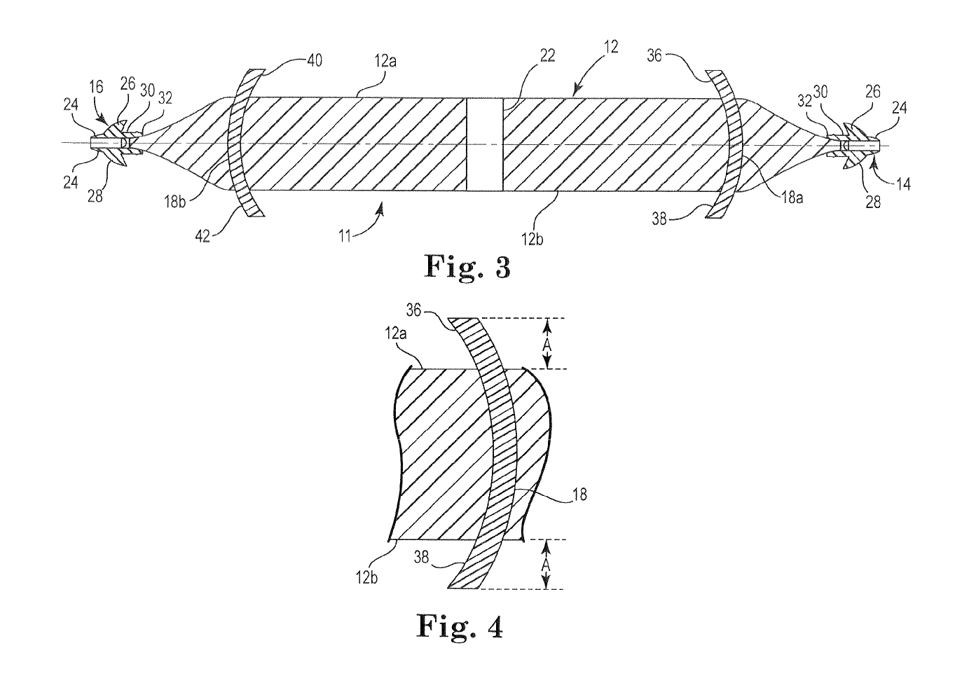

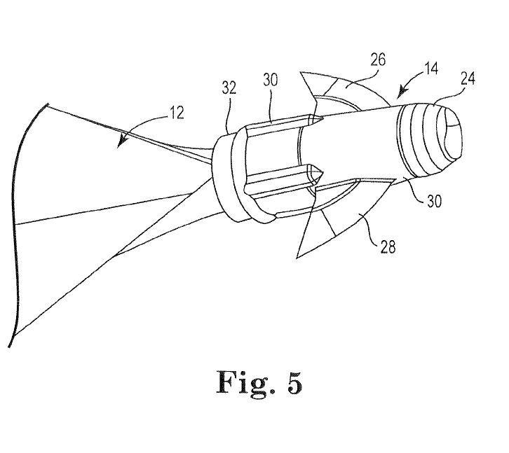

The extension portion 12 generally extends between and is integrated or otherwise coupled with the two end tips 14, 16. In alternative embodiments, a single tip, or no tip at all, could be implemented with the present invention. In those embodiments having one or more tips 14, 16, the tips can include an end portion 24, a first anchoring tine 26, a second anchoring tine 28, a body portion 30, and a coupling portion 32, as shown in FIGS. 3 and 5. Further, the anchoring tines 26, 28 can be angled, rounded, linear, or take on a myriad of other shapes and configurations. The coupling portion 32 is adapted for fixation with the extension portion 12. Fixation can be achieved by molding, mateable engagement, clipping, bonding, or other like techniques. The overall dimensions of the implant device 11 may be 6 to 15 cm in length. Other proportional and dimensional embodiments may be employed without deviating from the spirit and scope of the present invention.

In certain embodiments, the one or more intermediate anchors 18 can include a plurality (e.g., two or more) of intermediate anchors 18a . . . 18n. The anchors 18 can be constructed of polypropylene, polyglycolic acid (PGA), polylactide (PLA), copolymers of PGA and PLA, silicone, or any other material known by those skilled in the art, biodegradable or non-biodegradable. As such, the anchors 18 can be generally rigid, hingeable, flexible or otherwise deformable to facilitate placement and fixation within the pelvic region.

As particularly illustrated in FIGS. 2-4, the implant device 11 can include two intermediate anchors 18a, 18b. The intermediate anchors 18a, 18b can be generally arcuate in shape, with end regions 36, 38 and 40, 42, respectively, extending out from the extension portion 12. For example, FIG. 4 depicts a length A of the end regions extending out, e.g., generally transverse, from the generally longitudinal sides 12a, 12b of the extension portion 12. Further, the one or more intermediate anchors 18 can be configured or positioned such that they extend out (e.g., generally transverse) from other surfaces of the extension portion 12, such as the top, bottom, and like planes or surfaces of the extension portion 12. Such anchor configurations can be included in lieu of or in addition to anchors 18 extending out from the longitudinal sides 12a, 12b. In various embodiments, the intermediate anchors 18a, 18b can be generally V-shaped (FIG. 1), U-shaped, straight, undulating, etc. The end regions can be straight, angled, rounded, jagged or take on other shapes and configurations to facilitate attachment, fixation and/or retention to tissue. In addition, other arcuate, linear or angled shapes can be implemented for the overall design and shape of the anchors 18. Further, the anchors 18 can be placed at, attached to, or proximate the tip portions 14, 16, or they can be placed in any desired location along a length of the extension portion 12. Various denution surfaces, protrusions, fibers, textures and the like can be included along portions of the intermediate anchors 18 (e.g., the end regions 36-42) to promote tissue disruption and/or fixation.

The sling extension portion, tips and anchors can exhibit desirable “adjustability” or “positionability” features, without the need for a length-adjusting mechanism. Each tip or respective intermediate anchor of the implant device can be placed within a pelvic tissue such as tissue of the obturator foramen, with properties of the tips and anchors (e.g., dimensions, pullback force, number of lateral extensions) and the implant (dimensions such as length between the tips and corresponding anchors) being sufficient to allow placement at tissue on one or both sides of the pelvic region, while the sling extension portion of the implant supports the urethra, bladder neck, vaginal tissue, etc. Desired positioning of the implant, the proximity to the supported tissue (e.g., urethra), or the amount of supportive force placed on the supported tissue, can be achieved by selectively placing the tips and intermediate anchors.

A fixed length of implant material can be of a single piece of material (integral), or may be of multiple pieces secured together. Pieces of the implant 11 can be sewn or otherwise secured together, or pieces of synthetic material may be sewn or otherwise secured to a biologic material. For instance, as shown in FIG. 2, the intermediate anchors 18 can be attached to (e.g., bonded to and/or sewn or interwoven with the strands or filaments of) the extension portion 12. Various other bonding or attachment methods and techniques can be employed as well.

The implant system 10 can further include an insertion or deployment tool 50. Various types of insertion tools are known, and these types of tools and modifications thereof can be used according to this description to install an implant. Examples of useful tools include those described and depicted in previously-incorporated PCT Patent Publication No. 2008/057261. In one embodiment, the tool 50 generally includes a thin elongate needle or shaft portion 52 that attaches to a handle 54. A distal end 56 of the needle 52 can be adapted to engage one of the tips 14, 16. The tip allows the needle to push the sling implant 11 through a tissue passage and insert the extension portion 12 and corresponding anchors 18 within, confronting or along tissue of the pelvic region. Other embodiments can utilize an insertion tool 50 including a catheter delivery system, wherein at least portions of the implant 11 are adapted for positioning and deployment from a shaft of the catheter.

Exemplary insertion tools for use according to the invention can be similar to or can include features of tools described in the above-referenced patent publications. For example, the insertion tool 50 may be used to place the implant 11 and anchors 18 at tissue within the pelvic region through a tissue path that does not extend to an external incision, e.g., transvaginally. The insertion tool can be designed, shaped, and sized to include an elongate inserter or needle that may be straight or that may be curved in two or three dimensions, that can be inserted through a vaginal incision (for female anatomy) or through a perineal incision (for male anatomy), and to extend from that incision to a pelvic tissue location for placement of the extension portion 12, end or tip portions 14, 16, and intermediate anchors 18a, 18b. As such, the extension portion 12 can be positioned to cradle, press against or otherwise support the tissue.

According to certain methods of the invention, the tip portions 14, 16 and/or the anchors 18 may be placed into pelvic tissue that is a fibrous tissue such as muscle, ligament, or tendon, with specific examples including the arcus tendineus, the obturator internus muscle, the levator ani, and the sacrospinous ligament. The end regions 36-42, or portions thereof, of the intermediate anchors 18 can be designed for implantation within the fibrous tissue at an orientation that places the lateral extending end regions 36-42 in a direction that is non-parallel to the fibers of the fibrous tissue. As such, the end tips 14, 16 can provide an initial placement fixation to the tissue, with the intermediate anchors 18 further securing or retaining (e.g., secondary fixation) the implant 11 within or against the tissue.

One example of a method according to the invention is a method of treating urinary incontinence by surgical implantation of a urethral sling implant 11 along a tissue path that extends from a region of the urethra to the obturator foramen. These methods can advantageously involve only a single incision (a vaginal incision in a female or a perineal incision in a male) and can exclude the need for any additional incision. The elongate urethral sling 11 is attached at tissue of the opposing obturator foramen by the respective tip portions 14, 16, with the extension portion 12 positioned to pass below the urethra to support the urethra. The intermediate anchors 18a, 18b can be fixated or anchored to the tissue at or proximate the tip portions 14, 16 (e.g., the obturator foramen).

All patents and publications referenced herein are hereby incorporated by reference in their entireties.

It will be understood that certain of the described structures, functions and operations of the above-described preferred embodiments are not necessary to practice the present invention and are included in the description simply for completeness of an exemplary embodiment or embodiments. It will also be understood that there may be other known structures, functions and operations ancillary to the typical surgical procedures that are not disclosed, but that can be implemented to practice the present invention. It is, therefore, to be understood that within the scope of the appended claims, the invention may be practiced other than as specifically described without actually departing from the spirit and scope of the present invention.

What is claimed is:

1. A pelvic implant device, comprising:
a mesh extension portion having a first end, a second end, a first longitudinal side and a second longitudinal side;
a first tip portion provided in communication with the first end of the mesh extension portion and adapted for tissue fixation; and
at least one intermediate anchor having a first end portion, a second end portion and a spanning portion, with the spanning portion spanning across a portion of the mesh extension portion such that the first end portion extends out from the first longitudinal side of the mesh extension portion and the second end portion extends out from the second longitudinal side of the mesh extension portion.

2. The device of claim 1, further including a second tip portion provided in communication with the second end of the mesh extension portion.

3. (canceled)

4. The device of claim 1, wherein the first tip portion includes a body portion and one or more extending tines.

5. The device of claim 1, wherein the at least one intermediate anchor includes at least two intermediate anchors, with the spanning portion of each of the at least two intermediate anchors spanning generally across a portion of the mesh extension portion such that the first end portion of each of the at least two intermediate anchors extends out from the first longitudinal side of the mesh extension portion and the second end portion of each of the at least two intermediate anchors extends out from the second longitudinal side of the mesh extension portion.

6. The device of claim 1, wherein at least the spanning portion of the at least one intermediate anchor is generally arcuate.

7. The device of claim 1, wherein at least the spanning portion of the at least one intermediate anchor is generally V-shaped.

8. The device of claim 1, wherein at least a portion of the spanning portion of the at least one intermediate anchor is coupled to the mesh extension portion.

9. The device of claim 1, wherein at least a portion of the spanning portion of the at least one intermediate anchor is bonded to the mesh extension portion.

10. A pelvic implant system, comprising:
an implant device including;
an extension portion and at least one fixating tip portion;
at least one intermediate anchor having a first end portion, a second end portion and a spanning portion, with the spanning portion spanning generally across a portion of the extension portion such that the first end portion extends out from a first longitudinal side of the extension portion and the second end portion extends out from a second longitudinal side of the extension portion; and
an insertion tool having a handle portion and a shaft portion, with a distal end of the shaft portion adapted to selectively engage with the implant device to facilitate deployment of the implant device.

11-12. (canceled)

13. The system of claim 10, wherein the at least one intermediate anchor includes at least two intermediate anchors, with the spanning portion of each of the at least two intermediate anchors spanning generally transverse across a portion of the extension portion such that the first end portion of each of the at least two intermediate anchors extends out from the first longitudinal side of the extension portion and the second end portion of each of the at least two intermediate anchors extends out from the second longitudinal side of the extension portion.

14. The system of claim 10, wherein at least the spanning portion of the at least one intermediate anchor is generally arcuate.

15. The system of claim 10, wherein at least a portion of the spanning portion of the at least one intermediate anchor is coupled to the extension portion.

16. The system of claim 10, wherein the extension portion is constructed of a mesh material.

17. The system of claim 10, wherein the shaft portion is adapted to selectively engage with the at least one fixating tip portion.

18-19. (canceled)

20. The system of claim 10, wherein the insertion tool includes a catheter delivery device.

21. A method of implanting a device in the pelvic region of a patient, comprising:
providing an implant device including an extension portion, at least one fixating tip portion and at least one intermediate anchor, the at least one intermediate anchor having a first end portion, a second end portion and a spanning portion, with the spanning portion spanning across a portion of the extension portion such that the first end portion extends out from a first longitudinal side of the extension portion and the second end portion extends out from a second longitudinal side of the extension portion;
providing an insertion tool having a handle portion and a shaft portion, with a distal end of the shaft portion adapted to selectively engage with the implant device;
inserting the implant device transvaginally into the pelvic region of the patient with the insertion tool; and
deploying the implant device such that the at least one fixating tip portion is engaged within tissue and the at least one intermediate anchor is positioned to provide anchoring of the implant device with tissue, such that the extension portion provides support for target pelvic tissue.

22. The method of claim 21, wherein providing the implant device having at least one fixating tip portion includes providing the implant device having two fixating tip portions.

23. (canceled)

24. The method of claim 21, wherein providing the implant device having at least one intermediate anchor includes providing the implant device having at least two intermediate anchors.

25. The method of claim 21, wherein the spanning portion of the at least one intermediate anchor is generally arcuate.

26. The method of claim 21, wherein providing the implant device having the extension portion includes providing the extension portion constructed of a mesh material.

27-28. (canceled)
